(19) United States
(12) Patent Application Publication
(10) Pub. No.: US 2009/0105804 A1
(43) Pub. Date: Apr. 23, 2009

(54) Medical implanting devices provided with anti-trombogenic coating and method for obtaining of such coating

(75) Inventors: Edward G. Shifrin, Raanana (IL); Nikolai Sedelnikov, Moscow (RU)

Correspondence Address: MINTZ LEVIN COHN FERRIS GLOVSKY & POPEO, ONE FINANCIAL CENTER, BOSTON, MA, 02111, US

(73) Assignee: S.V.SE 200 (1995) LTD., Raanana (IL)

(21) Appl. No.: 12/151,931

(22) Filed: May 9, 2008

Related U.S. Application Data
(60) Provisional application No. 60/917,092, filed on May 10, 2007.

(51) Int. Cl. A61F 2/06

(52) U.S. Cl. 623/1.13; 623/1.15; 623/1.42; 623/1.46

(57) ABSTRACT

A medical implantable device for deployment within a vessel of a mammal patient is disclosed. The device has at least one surface, which might come in contact with blood, said at least one surface being coated by a biocompatible anti-trombogenic coating. The anti-trombogenic coating being presented in a thermodynamic non-equilibrium labile state defined by a surface energy favorable for immobilizing of albumen thereon while preventing adhesion of thrombogenic proteins thereto.

### CROSS-REFERENCE TO RELATED APPLICATIONS

The present application claims priority to U.S. Provisional Application No. 60/917,092, filed May 10, 2007, and incorporates its entire disclosure hereby reference.

### FIELD OF THE INVENTION

The present invention refers to medical implantable devices deployable inside a vessel within a body of a mammal patient. More particularly, the invention refers to medical implantable devices provided with anti-trombogenic coating covering those surfaces of the implantable devices that contact with blood to prevent blood clotting and hyperplasia on these surfaces. The present invention concerns also a method for obtaining of such anti-trombogenic coating.

### BACKGROUND OF THE INVENTION

Known in the art are many artificial medical devices deployable inside a body of a mammal patient and containing at least one surface contacting with blood.

These devices are implantable in a vascular or endoluminal location within the body of the patient to maintain a lumen open at that location. Among such devices are implantable cardiovascular devices, such as stents, grafts, stent-grafts, shunts, patches, heart valves, attachment cuffs, etc.

These artificial devices have been developed to be surgically implantable within the body of the patient to replace damaged or defective natural vessel or valve. Such devices are made from materials selected for their ability to be compatible with the patient's body, to handle the requirements of fluid pressures in the affected vessel or valve, and to provide attachment sites for the anchoring of sutures and the formation of scar tissue. Among materials used for manufacturing of implantable medical devices are e.g. polytetrafluoroethylene or PTFE (known also under the registered trademark “Teflon”) and polyethylene glycol terephthalate (known also under the registered trademark “Dacron”). Both materials are especially suitable for manufacturing of knitted, woven or braided cardiovasacular devices like implants, grafts, or attachment cuffs. Another material, which is used for manufacturing of shunts, grafts and patches is an expanded microporous polytetrafluoroethylene or ePTFE (known also under the registered trademark “Gore-Tex”).

Examples of catheters, heart valves, or plastic reconstructive surgical material, to be at least partially embedded in an implantation site in a soft organic tissue of a living organism are shown and described in U.S. Pat. No. 5,219,361 (von Recum and Campbell) and U.S. Pat. No. 5,011,494 (von Recum and Campbell). The soft tissue implant devices include a body defining surface layer extending over the portion of the body contacting the organic tissue. The surface layer defines a three-dimensional pattern with an exterior surface having a plurality of spaces and a plurality of solid surface portions. The spaces have a mean bridging distance ranging from greater than 1.0 micron to less than 4.0 microns and the solid surface portions have mean breadths ranging from 0.10 micron to 2.0 microns.

The presence or formation of thromboses or blood clots is of significant concern in any surgical procedure, and is also a most serious problem when there are used implantable cardiovascular devices, e.g. arterial-venous shunts, grafts, patches or artificial heart valves. Clotting frequently occurs in dialysis shunts or grafts and this requires their often removal, cleaning and surgical re-implantation. The formation and dislodging of a clot may result in the occlusion or blocking of a blood vessel and interrupting the life-giving flow of blood to major organs of the body. Formation of thromboses in surgically implanted arterial or venous grafts may occur because of such factors like the woven, porous nature of the graft material which may attract blood platelets or debris contained in the blood stream. The graft's chemical composition, its compliance, and/or its electro-negativity, each of which may evoke a different tissue reaction which eventually may also contribute to thrombosis. See, for example, Greisler, et al., “Plasma Polymerized Tetrafluoroethylene/Polyethylene Terephthalate Vascular Prostheses”, Arch. Surg. Vol. 124, pp. 967-972 (August 1989). This article teaches that once a mass of detritus reaches a significant weight and size, it may adhere to the wall of the blood vessel, progressively blocking the vessel, or it may be dislodged by the flow of blood through the blood vessel and then travel until it encounters a blood vessel having a diameter less than that of the thrombus, thus eventually causing a blockage.

For the prevention of formation of thromboses in vascular shunts, grafts and artificial heart valves various methods or contrivances have been used, which may, in its turn, limit thrombogenic properties of such devices.

Examples of vascular shunts are shown and described in U.S. Pat. No. 4,167,045 (Sawyer).

Sawyer teaches a vascular shunt made from Dacron (Registered U.S. Trademark), coated with glutaraldehyde-polymerized proteins, aluminum or other substances. Sawyer also teaches that early attempts to use rigid, gold tubes as vascular shunts were unsuccessful.

U.S. Pat. No. 4,355,426 (MacGregor) describes the use of metallic porous vascular grafts for prevention of formation of thromboses.

In other attempts to limit formation of thromboses coatings were applied to the grafts.

U.S. Pat. No. 4,718,907 (Karwoski et al.) teaches using a fluorinated coating applied electrically to the surfaces of interwoven fabric tube.

U.S. Pat. No. 4,265,928 (Braun) describes a thin coating of an ethylene-acrylic acid copolymer.

The use of homogeneous synthetic materials, e.g. “Teflon”, “Dacron” or “Gore-Tex” appeared to be more successful. However, the porous structure of these materials may itself cause formation of thromboses since it may serve as a trap for the debris present in the blood stream, thus creating the centers of formation and propagation of thromboses. The graft's chemical composition and/or its electro-negativity may also contribute to thrombosis.

In order to limit the formation of thrombosis at least one surface of the implantable device contacting with the blood may be provided with a metal coating which either fills the pores of the surface or coats the whole surface.

U.S. Pat. Nos. 4,557,975 and 4,720,400 (Manisso) describe the application of coatings, including metal coatings, to synthetic non-woven fabric made from microporous polytetrafluoroethylene (ePTFE). This material is characterized by a microstructure consisting of nodes interconnected by fibrils. A continuous interporous metal coating encapsulates the nodes and fibrils of the PTFE while maintaining substantial porosity. The encapsulation of the nodes and fibrils is achieved by immersion the fabric into a liquid solution and chemical deposition of the metal from that liquid.

U.S. Pat. Nos. 5,464,438 and 5,207,706 (Menaker) describe implantable vascular prostheses like grafts, shunts, patches or valves, made from synthetic, woven fibers coated by a thin layer of metallic gold to form a non-thrombogenic surface. Methods of manufacture are also disclosed. The coating is applied to the inner wall of the vascular prosthesis by vapor deposition or sputtering to coat the fibers without blocking or bridging the interstices formed by the fiber intersection. All these prostheses use the therapeutic properties of gold since the body's long-term tolerance to the presence of gold has been recognized by the medical profession. The using of gold in the cardiovascular prostheses is known to prevent bacterial infection, however the use of a continuous gold coating was not suggested for creating a non-thrombogenic surface for permanent implantation.

The closely related (by the choice of material and method of coating) to the present invention are “Soft tissue implants” described in U.S. Pat. Nos. 4,871,366 and 4,846,834 (von Recum and Cooke). These patents describe soft tissue implants comprising a flexible main body portion and a tissue-facing surfaces covered by a thin layer of pure titanium. The patents refer also to a method of promoting tissue adhesion to a soft tissue host of the tissue-facing surfaces of a soft tissue implant provided with a strip of polyethylene terephthalate velour. The method comprises the following steps: cleaning the strip with a low-residue detergent and rinsing same with fresh distilled water; refluxing the strip in distilled water for one hour at a temperature of less than 30 degree C.; drying the strip at a room-temperature in a dessicator for several days; sterilizing the strip and packaging same; degassing the strip and storing same in a dust-free environment; removing the strip from the packaging and mounting the strip in the vacuum evaporator; evacuating the vacuum evaporator to a certain degree of vacuum; evaporating the titanium by direct resistance heating; coating the strip with a layer of pure titanium having thickness of one micron; and re-sterilizing and implanting the titanium-coated strip into the tissue host.

There are known in the art also “Vascular and endoluminal stents with iridium oxide coating” according to U.S. Pat. No. 5,980,566 by Eckhard Alt and Lawrence J. Stotts. The patent describes a vascular stent adapted to be implanted in a blood vessel of a human patient to enhance the flow of blood through the vessel. The stent is configured as an elongate biocompatible metallic member having cylindrical shape, which sidewall is provided with a pattern having multiple openings. The stent has an insertion outer diameter that is sufficiently small to enable insertion of the stent into and advancing through a portion of the vascular system of the body towards a pre-selected location within a coronary artery. The sidewall has a thin coating of iridium oxide covering substantially entire exposed sidewall's surface, including the outward-facing surface between openings, the edges of the openings, the inward-facing surface between openings, and the edge of each of the open ends. The coating is of substantially uniform thickness and serves to reduce irritation of tissue of the inner lining of the vessel wall with which the outward-facing surface of the stent comes into contact. The tissue may project as well into the openings in the sidewall to contact with the edges thereof. The iridium oxide coating is provided with a biodegradable carrier of drugs applied thereto for beneficial localized action, e.g. by incorporating into the carrier along the inward-facing surface an anticoagulant drug to reduce attachment of thrombi with blood flow through the stent. Although it may be composed of multiple layers, the iridium oxide coating is sufficiently thin and flexible to resist flaking during deployment, and the core member has sufficient rigidity to resist collapse.

Furthermore there are known “Non-thrombogenic implantable devices” as described in International Publication WO 0158504. These devices were devised to cope with a phenomenon associated with the formation of thromboses in the presence of electrostatic charges on the surface exposed to the recipient's bloodstream. These electrostatic charges facilitate adsorption of blood elements onto the surface exposed to the bloodstream, which, in turn, causes formation of thromboses. Prosthetic material used for manufacturing of implants has metal coating defined by micro-scale structure which is substantially amorphous or quasi-amorphous. This structure of the coating in combination with a low, and preferably non-homogeneous thickness enables to retain an almost zero or even slightly negative electrode potential during exposure to the bloodstream, or to air, water etc. Accordingly, such a substantially amorphous thin metal coating provides non-thrombogenic or even anti-thrombogenic properties, since the elements contained in blood are either not encouraged to adsorb onto the coating surface, or indeed are actively repelled from the coating.

However, as the medical practice shows, this solution is still not enough for essential reduction of blood clotting risk and, moreover, for the elimination of hyperplasia especially in the case of distal anastomosis at a slow blood stream flow.

It is known also an implant described in U.S. Pat. No. 6,110,204. The patent relates to an implant for use in a human body. An implant substrate is coated with a material containing some metals, N, O, while 2-45% of the coating volume being formed by voids. The mechanism of anti-thrombogenic action is not disclosed.

### SUMMARY AND OBJECTS OF THE INVENTION

To sum up the previous solutions it could be stated as follows.

As a rule, in order to fight blood clotting, attempts are undertaken to prevent adsorption and activation of platelets and fibrinogen on the implant surface.

Many researchers looked for a suitable method to affix endothelium cells on the inner surface of artificial cardiovascular devices since it induces fast neointima escalating, which turns to be the same or almost the same layer that covers the original vessel from inside. On that layer no debris may be left.

Unfortunately, this approach is not suitable for implementation on industrial scale. Growing up of such layers requires long time and, besides, only live endothelium cells of a patient to whom the implantable device should be implanted can be used.

One can conclude that despite of numerous and various attempts to devise implantable devices which resist to blood clotting, there still exists a room for a new and improved solution, which combines advantages of the known approaches but is free of their disadvantages.

The main object of the present invention is to provide a new and improved medical implantable device having improved anti-trombogenic properties.

The other object of the invention is to provide medical implantable device having improved anti-trombogenic properties on account of a coating deposited on the inner surface of the device while the coating being capable to efficiently immobilize albumin thereon and to prevent adhesion of glycoprotein's and eventually to reduce adhesion and activation of thrombocytes.

Another object of the invention is to provide medical implantable device having a coating deposited on the inner surface of the device said coating being capable to promote formation and growth of neointima in vivo.

The yet another object of the present invention is to provide a new and improved implantable medical device having metal coating on its inner surface, which combines highly anti-thrombogenic properties in combination with elasticity and good surface adhesion which remain during mechanical deformation of the device, e.g. stretching or bending.

Still further object of the invention is to provide a method for manufacturing of implantable medical devices with improved anti-trombogenic properties while this method being implementable on industrial scale.

### LIST OF DRAWINGS

FIG. 1 depicts an example of a structure of a coating of the present invention.

### DETAILED DESCRIPTION OF THE INVENTION

In accordance with the present invention there is provided medical implantable device having improved anti-trombogenic properties by virtue of a dedicated biocompatible coating applied thereto. The coating is applied to an inner surface of the device, i.e. to that surface, which contacts blood.

In accordance with the invention the improved anti-trombogenic properties are achieved when the coating consists of a metal or of a metal containing substance and when the coating has thickness of 200-500 nm and when the structure of the coating comprises a plurality of substantially separate particles, having nearly spherical shape with a diameter of 10-200 nm.

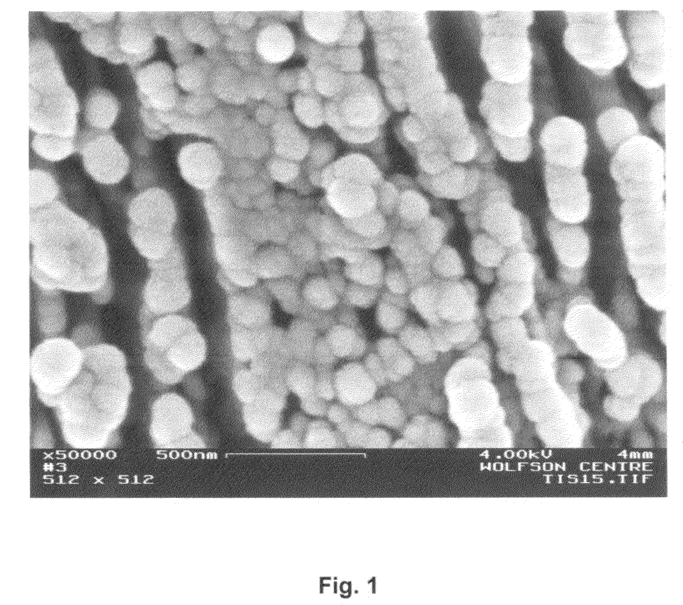

An example of a coating in accordance with the present invention is shown in FIG. 1, which depicts a picture obtained by high resolution scanning electron microscopy (HR SEM).

In this FIGURE the coating is made of Titanium and it is applied to a medical implantable device made of ePTFE. It should be borne in mind however that this is only one example and that within the invention are also coatings made of other metals or metal containing substances or compounds as well as implantable devices made of materials different from ePTFE.

To achieve the improved anti-trombogenic properties it is preferable that the coating exists in its active form which means that the coating surface has plurality of unblocked centers, which readily immobilize albumen thereon.

In practice in order to obtain the coating in active form it is deposited in vacuum. It is also preferable if before the coating is deposited the inner surface of the device is treated in order to increase its roughness. An example of a suitable treatment is ion etching or chemical etching.

The active form of the coating can be achieved when it is in a non-equilibrium labile state, i.e. when it is in a frozen equilibrium. If the coating is made of Titanium such active form would be high-temperature polymorphous phase defined by a body-centered cubic structure. It is preferable that the surface texture of the coating would correspond to a most close-packed crystallographic plane. For a coating which presents as Titanium body-centered cubic phase such most close-packed plane would be the plane (110).

When the coating is in active form as mentioned above it has increased surface energy which renders favorable conditions for adsorption and immobilization of albumen. The immobilized albumen prevents adhesion of fibrinogen and platelets, which are thrombogenic and by virtue of this provision improved anti-trombogenic properties are eventually achieved.

The texture of the coating can be analyzed by a known in the art suitable X-ray methods, for example, by using reversed pole figures.

Despite Titanium is the preferred coating material, various other inorganic or organic materials, e.g. refractory metals, noble metals, compounds of metals, carbon, synthetic or natural substances, ceramics, etc., can be used as well.

The suitable material for implantable device can be any organic or inorganic material known in the art, e.g. Teflon, Dacron, Gore-Tex, Stainless steel etc.

The basic method for obtaining anti-trombogenic coating of the invention is physical vapor deposition, e.g. magnetron sputtering, ion beam deposition or chemical vapor deposition.

In practice magnetron sputtering is preferable method, since it provides desirable results and can be conveniently implemented on industrial scale.

Depending on the material, from which the implantable device is made, it might be desirable before sputtering to carry out a preliminary treatment of the surface to be coated. The treatment might be required if the coating is deposited on metallic surface.

The preliminary treatment comprises cleaning of the surface followed by creating a rough micro relief thereon. The suitable micro relief after treatment can be defined by the following parameters according to ASME 46.1: Roughness average Sₐ of about 0.2-0.4 and Ten Point Height S_{z} of about 0.32-0.64.

Now with the aim of the following non-limiting examples a process for depositing anti-trombogenic coating on an implantable medical device will be described.

In the following examples the inner surface of the implantable device, which faces the bloodstream and on which the coating is deposited will be referred-to as a substrate.

### EXAMPLE 1

A medical implantable device, e.g. a vascular graft made of ePTFE is placed in an ultrasonic bath containing a solution of low residue detergent in distilled water and is ultrasonically cleaned. Rinsing by freshly distilled water follows this step, and the graft is then dried in a desiccator at ambient temperature. Then the dried graft is placed in a vacuum chamber with a residual pressure of about 0.1 Pa and the chamber is heated up to 100-150° C. Ionic etching of the substrate is then performed at a residual pressure of Ar between 0.1-1.0 Pa. The purpose of ionic etching is creating a juvenile (virgin) surface on the substrate. Then Ti coating is deposited onto the etched substrate by a sputtering process. The sputtering process is typically performed with argon-oxygen plasma at pressures ranging from about 0.5 Pa to about 10.0 Pa. During the sputtering the Ar:O₂ ratio is maintained within 2÷0.5.

The optimal value of the power density during sputtering typically is maintained between 0.5÷2 Watt/cm2. The sputtering step is typically carried out at a potential from approximately 200 Volt to approximately 500 Volt. A target made of Titanium Grade 1 or 2 according to ASTM B265 is used as a source of ions of Titanium. The sputtering process lasts about 20 seconds.

The coated by the above procedure grafts as well as uncoated grafts were implanted in dogs as abdominal aorta left common iliac bypass. In order to evaluate the response of the grafts to blood stream the dogs were observed during several weeks and then successively sacrificed after different periods of time. A histopathology study was carried out. The study included examination of sutures and evaluation of the implanted coated and uncoated grafts, including observation of presence or absence of thrombosis and neonintima response. The inner surface of the coated grafts was smooth and did not show thrombosis or signs of fibrin or platelet aggregation. No thrombi were seen. The histology study of the coated grafts revealed in vivo growth of neointima with fresh endothelium cells.

In contrast to the coated grafts the uncoated grafts were occluded with thrombosis.

### EXAMPLE 2

A medical implantable device, e.g. a vascular stent made of steel 316L is ultrasonically cleaned as previously described in Example 1.

The cleaned stent is placed in a vacuum chamber where it is treated by a cyclic treatment comprising combination of sputtering-annealing cycles followed by oxidation-reduction cycles.

The purpose of this cyclic treatment is imparting a rough micro relief to the substrate. The cyclic treatment comprised 2-10 cycles of ion etching by argon-ion sputtering (500 eV, 20 μA/cm2, 15-30 min) followed by annealing (450-600° C., 1-60 min). This cyclic treatment was complemented by cleaning, which included heating the stent to 500° C. and exposing it to oxygen and after that oxygen elimination with acetylene. Upon completing the treatment step Titanium coating was deposited on the substrate by sputtering. The sputtering step was carried out during 20 seconds at a power density between about 8.0 Watt/cm2 to about 10.0 Watt/cm2.

It has been empirically revealed that medical implantable devices provided with the coating deposited as described in the above examples have improved anti-trombogenic properties.

In particular it has been observed that albumen covers the coating while fibrinogen and thrombocytes do not adhere. Furthermore formation of neointima in vivo has been observed during first four weeks after implantation.

One skilled in the art would appreciate that the method of manufacturing of such coatings which comprises etching and sputtering can be easily realized on industrial scale.

The features disclosed in the foregoing description, and/or in the foregoing drawings and/or following claims both separately and in any combination thereof, be material for realizing the present invention in diverse forms thereof.

When used in the following claims, the terms “comprise”, “include”, “have” and their conjugates mean “including but not limited to”.

As used herein, the term “medical implantable device” refers to a device intended for placement and securing in a body of a mammal human or an animal patient.

Non-limiting examples of such devices are stents, grafts, stent-grafts, shunts, patches, heart valves, attachment cuffs, etc.

As used herein, the term “vessel” refers to any hollow vessels or ducts or cavities available in a mammal body. Non-limiting examples of such passage are arteries, veins, intestines, valves, etc.

The scope of the invention is defined by the appended claims.

1. A medical implantable device for deployment within a vessel of a mammal patient, said device having at least one surface coming in contact with blood, said at least one surface being coated by a biocompatible anti-trombogenic coating existing in a thermodynamic non-equilibrium labile state defined by a surface energy sufficient for immobilizing of albumen thereon while preventing adhesion of thrombogenic proteins thereto.

2. The medical implantable device of claim 1, in which said coating presents as a non-equilibrium high temperature crystalline phase having a texture corresponding to a most closed packed crystallographic plane.

3. The medical implantable device of claim 1, in which said coating has a thickness of 200-500 nm and said coating being defined by a structure comprising a plurality of separate particles having a size of 10-200 nm.

4. The medical implantable device of claim 3, in which said particles are nearly spherical.

5. The medical implantable device of claim 4, in which said coating is made of a material selected from the group consisting of metals, compounds of metals, metal alloys, metal containing substances, ceramics and organic materials.

6. The medical implantable device of claim 5, in which said at least one surface is made of a material selected from the group consisting of organic materials and inorganic materials.

7. The medical implantable device of claim 6, in which said organic materials are selected from the group consisting of polyurethane, copolymers of polyurethane, derivatives of polyurethane, polyethylene glycol terephtalate, poly-tetrafluoroethylene and expanded microporous polyfluoroethylene.

8. The medical implantable device of claim 6, in which said inorganic material is metallic material.

9. The medical implantable device of claim 7, in which said coating is made of Titanium and said at least one surface, is made of expanded microporous polyfluoroethylene.

10. The medical implantable device of claim 8, in which said coating is made of Titanium and said at least one surface is made of stainless steel.

11. The medical implantable device of claim 9, in which said coating is present as a body-centered cubic phase having most close-packed crystallographic plane (110).

12. The medical implantable device of claim 1, in which said device is a stent.

13. The medical implantable device of claim 1, in which said device is a graft.

14. A method of providing a medical implantable device with a biocompatible anti-trombogenic coating, comprising:
providing a medical device implantable within a body of a mammal patient said device having at least one surface coming in contact with blood;
pretreatment of the at least one surface to impart thereto a roughness defined by the Roughness average Sₐ of about 0.2-0.4 and Ten Point Height S_{z} of about 0.32-0.64;
depositing on the at least one surface of the device the biocompatible anti-thrombogenic coating said coating existing in a thermodynamic non-equilibrium labile state defined by a surface energy sufficient for immobilizing of albumen thereon while preventing adhesion of thrombogenic proteins thereto.

15. The method of claim 14, in which said coating presents in a non-equilibrium crystalline phase having a texture corresponding to a most closed packed crystallographic plane.

16. The method of claim 15, in which said coating has a thickness of 200-500 nm and said coating being defined by a structure comprising a plurality of separate particles having a size of 10-200 nm.

17. The method of claim 16, in which said particles are nearly spherical.

18. The method of claim 17, in which said coating is made of a material selected from the group consisting of metals, compounds of metals, metal alloys, metal containing substances, ceramics and organic materials.

19. The method of claim 18, in which said at least one surface is made of a material selected from the group consisting of organic materials and inorganic materials.

20. The method of claim 19, in which said organic materials are selected from the group consisting of polyurethane, copolymers of polyurethane, derivatives of polyurethane, polyethylene glycol terephtalate, poly-tetrafluoroethylene and expanded microporous polyfluoroethylene.

21. The method of claim 20, in which said pretreatment comprises ion etching.

22. The method of claim 21, in which said pretreatment comprises ion etching by sputtering in Argon.

23. The method of claim 21, said depositing comprises any gas vapor deposition method selected from the group consisting of physical vapor deposition and chemical vapor deposition.

24. The method of claim 23, in which said physical vapor deposition is sputtering of Titanium in Argon-Oxygen plasma by using a target made from Titanium of grade 1 or 2 according to ASTM B265.

25. The method of claim 24, in which the sputtering is carried out for about 20 seconds at a voltage of 200-500 Volts, at a pressure of 1-3 Pa, and at a ratio between Argon and Oxygen of 0.5-1.

26. The method of claim 25, in which said at least one surface is made of expanded microporous polyfluoroethylene and the sputtering is carried out at a power density of 0.5-2 Watt/square centimeter.

27. The method of claim 21, in which said at least one surface is made of stainless steel and said pretreatment comprises 2-10 cycles of ion etching followed by annealing and oxidation-reduction.

28. The method of claim 27, in which said depositing comprises any gas vapor deposition method selected from the group consisting of physical vapor deposition and chemical vapor deposition.

29. The method of claim 28, in which said physical vapor deposition is sputtering of Titanium in Argon-Oxygen plasma by using a target made from Titanium of grade 1 or 2 according to ASTM B265.

30. The method of claim 29, in which the sputtering is carried out at a voltage of 200-500 Volts, at a pressure of 1-3 Pa, at a ratio between Argon and Oxygen of 0.5-1, and at a power density of 8-10 Watt/square centimeter.
